(19) United States
(12) Patent Application Publication
(10) Pub. No.: US 2026/0094274 A1
(43) Pub. Date: Apr. 2, 2026

(54) SYSTEMS AND METHODS FOR PREDICTING PANCREATIC DUCTAL ADENOCARCINOMA

(71) Applicant: CEDARS-SINAI MEDICAL CENTER, Los Angeles, CA (US)

(72) Inventors: Debiao Li, South Pasadena, CA (US); Stephen Jacob Pandol, Los Angeles, CA (US); Touseef Ahmad Qureshi, Los Angeles, CA (US); Sehrish Javed, Los Angeles, CA (US); Lixia Wang, Los Angeles, CA (US); Srinivas Gaddam, Los Angeles, CA (US)

(73) Assignee: CEDARS-SINAI MEDICAL CENTER, Los Angeles, CA (US)

(21) Appl. No.: 19/112,186

(22) PCT Filed: Sep. 15, 2023
(86) PCT No.: PCT/US2023/074387

§ 371 Date: Mar. 14, 2025

Related U.S. Application Data
(60) Provisional application No. 63/407,290, filed on Sep. 16, 2022.

(51) Int. Cl. G06T 7/00; A61B 6/00; A61B 6/46; G06T 7/11; G06V 10/26; G06V 10/40; G06V 10/764; G06V 10/774; G16H 50/20

(52) U.S. Cl. CPC G06T 7/0016; A61B 6/54; G06T 7/11; G16H 50/20; A61B 6/461; G06T 2207/10081; G06T 2207/20021; G06T 2207/20076; G06T 2207/20081; G06V 10/26; G06V 10/40; G06V 10/764; G06V 10/774; G06V 2201/031

(57) ABSTRACT

A method for analyzing health of a pancreas of an individual includes receiving a computed tomography (CT) image of a pancreas of the individual; analyzing the CT image to determine a value of each of one or more radiomic features of the CT image; and based on the value of each of the one or more radiomic features of the CT image, determining a pancreatic ductal adenocarcinoma (PDAC) risk factor for the pancreas of the individual.

### CROSS-REFERENCE TO RELATED APPLICATIONS

This application claims the benefit of, and priority to, U.S. Provisional Patent Application No. 63/407,290, filed on Sep. 16, 2022, which is hereby incorporated by reference herein in its entirety.

### STATEMENT REGARDING FEDERALLY SPONSORED RESEARCH

This invention was made with government support under Grant No. CA260955 awarded by the National Institutes of Health. The government has certain rights in the invention.

### TECHNICAL FIELD

The present disclosure relates generally to systems and methods for predicting the occurrence of pancreatic ductal adenocarcinoma (PDAC), and more particularly, to systems and methods for analyzing CT images of the pancreas to predict the occurrence of PDAC.

### BACKGROUND

Early detection of Pancreatic Ductal Adenocarcinoma (PDAC) is complicated as PDAC remains asymptomatic until cancer advances to late stages when treatment is mostly ineffective. Stratifying the risk of developing PDAC can improve early detection as subsequent screening of high-risk individuals through specialized surveillance systems reduces the chance of misdiagnosis at the initial stage of cancer. Risk stratification is however challenging as PDAC lacks specific predictive biomarkers. Thus, new systems and methods for predicting the occurrence of PDAC are needed.

### SUMMARY

According to a first implementation of the present disclosure, a method for analyzing health of a pancreas includes receiving a computed tomography (CT) image of a pancreas of an individual; analyzing the CT image to determine a value of each of one or more radiomic features of the CT image, and, based on the value of each of the one or more radiomic features of the CT image, determining a pancreatic ductal adenocarcinoma (PDAC) risk factor for the pancreas of the individual.

According to a second implementation of the present disclosure, a method of training a machine learning model to analyze pancreas health includes obtaining a plurality of pre-diagnostic computed tomography (CT) images, each of the plurality of pre-diagnostic CT images showing a respective pancreas known to have subsequently developed a pancreatic ductal adenocarcinoma (PDAC); determining a value of each of a plurality of radiomic features in each of the plurality of pre-diagnostic CT images; and training the machine learning model to output a PDAC risk factor using the value of at least some of the plurality of radiomic features in each of the plurality of pre-diagnostic CT images, the PDAC risk factor being indication of whether a risk of a PDAC developing in a pancreas of an individual is high or low.

In some aspects of the second implementations, the method further includes obtaining a plurality of control CT images, each of the plurality of control CT images showing a respective pancreas known to have not subsequently developed a PDAC; determining a value of each of the plurality of radiomic features in each of the plurality of control CT images; comparing the values of the radiomic features in the pre-diagnostic CT images with the values of the radiomic features in the control CT images; and based on the comparing, identifying a first subset of radiomic features of interest from the plurality of radiomic features, wherein the at least some of the plurality of radiomic features used to train the machine learning model includes only the radiomic features in the first subset.

In some aspects of the second implementation, the pancreas is divided into a plurality of regions. The method can further include dividing each of the pre-diagnostic CT images into a plurality of regions corresponding to the plurality of regions of the respective pancreas of each of the plurality of pre-diagnostic CT images; determining the value of each of the plurality of radiomic features in each of the plurality of regions of each of the plurality of pre-diagnostic CT images; obtaining a plurality of diagnostic CT images, each of the plurality of diagnostic CT images corresponding to a respective one of the plurality of pre-diagnostic CT images and showing the respective pancreas after developing the PDAC; dividing each of the plurality of diagnostic CT images into a plurality of regions corresponding to the plurality of regions of the corresponding one of the plurality of pre-diagnostic CT images; and marking each region of each pre-diagnostic CT image as high-risk or low-risk based on comparing each region of each diagnostic CT image to the corresponding region of the corresponding pre-diagnostic CT image; and dividing each of the control CT images into a plurality of regions corresponding to the plurality of regions of the respective pancreas of each of the plurality of control CT images. Comparing the values of the radiomic features in the diagnostic CT images to the values of the radiomic features in the control CT images can include, for each respective region of the plurality of regions, comparing (i) the values of the radiomic features in the respective regions of the control CT images and the respective regions marked as low-risk in the pre-diagnostic CT images to (ii) the values of the radiomic features in the respective regions marked as high-risk in the pre-diagnostic CT images. Each radiomic feature in the first subset of radiomic features of interest has a change in value from (i) the respective regions of the control CT images and the respective regions marked as low-risk in the pre-diagnostic CT images to (ii) the respective regions of the pre-diagnostic CT images, where the change in value satisfies a predetermined threshold.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

The disclosure, and its advantages and drawings, will be better understood from the following description of representative embodiments together with reference to the accompanying drawings. These drawings depict only representative embodiments, and are therefore not to be considered as limitations on the scope of the various embodiments or claims.

FIG. 1 is a block diagram of a system for analyzing the health of a pancreas of an individual and/or for training a machine learning model to analyze the health of a pancreas of an individual, according to aspects of the present disclosure.

FIG. 2 is a flow chart of a method for analyzing the health of a pancreas of an individual, according to aspects of the present disclosure.

FIG. 3 is a flow chart of a method for training a machine learning model to analyze the health of a pancreas of an individual, according to aspects of the present disclosure.

FIG. 4 shows the data design of method for training a machine learning model to analyze the health of a pancreas of an individual, according to aspects of the present disclosure.

FIG. 5 shows a technique for grading different regions of a pancreas in a pre-diagnostic CT image of the pancreas, according to aspects of the present disclosure.

FIG. 6 shows an overview of a method for training a machine learning model to analyze the health of a pancreas of an individual, according to aspects of the present disclosure.

### DETAILED DESCRIPTION

Early detection of Pancreatic Ductal Adenocarcinoma (PDAC) is complicated as PDAC remains asymptomatic until cancer advances to late stages when treatment is mostly ineffective. Studies have reported that the pancreas undergoes local morphological changes in response to underlying biological evolution associated with PDAC development. Accurate identification of these changes can help stratify the risk of PDAC. Disclosed herein are systems and methods for performing radiomic analysis of precancerous pancreatic subregions using abdominal Computed Tomography (CT) images.

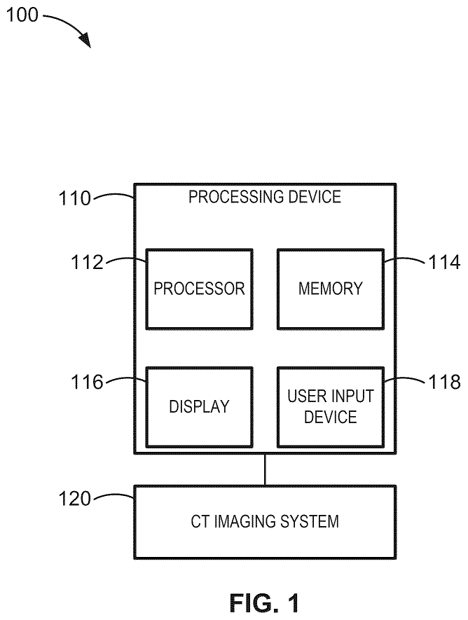

FIG. 1 illustrates a block diagram of system 100 that can be used to analyze pancreas health in a subject by examining CT images (and/or other types of images). The system 100 can include one or more processing devices 100, which can each include any one or more of a processor 112, a memory 114, a display 116, a user input device 118, and/or other components. The memory 114 can include machine-readable instructions for executing the methods disclosed herein, and/or other methods. The processor 112 can execute these instructions to implement these methods. The memory 114 can also store data associated with the methods, such as image data associated with CT images and/or other types of images.

The processing device 110 can include any suitable processing device, such as general purpose computer systems, microprocessors, digital signal processors, micro-controllers, application specific integrated circuits (ASICs), programmable logic devices (PLDs) field programmable logic devices (FPLDs), programmable gate arrays (PGAs), field programmable gate arrays (FPGAs), mobile devices such as mobile telephones, personal digital assistants (PDAs), or tablet computers, local servers, remote servers, wearable computers, or the like. The memory device 114 can include any suitable memory device and/or machine-readable medium that is capable of storing, encoding, and/or carrying a set of instructions for execution by a processing device and that cause the processing device to perform and/or implement any of the features discussed herein, including solid-state memories, optical media, magnetic media, random access memory (RAM), read only memory (ROM), a floppy disk, a hard disk, a CD ROM, a DVD ROM, flash memory, or other computer readable medium that is read from and/or written to by a magnetic, optical, or other reading and/or writing system that is coupled to the processing device, can be used for the memory or memories.

The display 116 can be used to display any information associated with the features disclosed herein, including the results of the classification analysis by the machine learning model. The display device 116 can be any known display technology, including but not limited to display devices using Liquid Crystal Display (LCD) or Light Emitting Diode (LED) technology. The user input device 118 can be used to allow the user to interact with the system 100 for any suitable purpose, including initiating, pausing, or terminating the analysis by the machine learning model; adjusting any parameters of the analysis, etc. In some implementations, the system 100 includes a CT imaging system 120 that generates the CT images that are used in the methods disclosed herein. The CT imaging system 120 can generally be any suitable type of CT imaging system. In other implementations, the system 100 does not include the CT imaging system 120, but instead receives CT images from an external source.

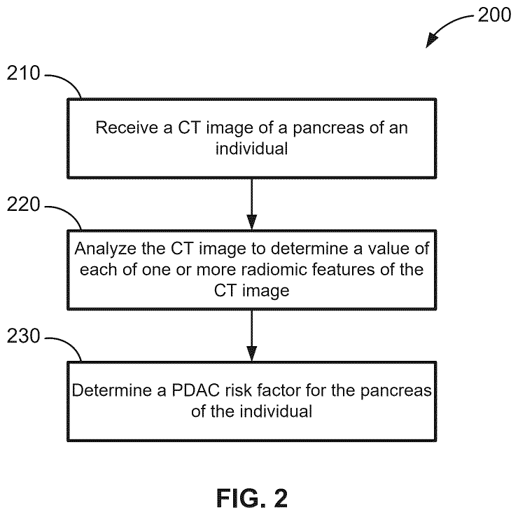

FIG. 2 shows a flow chart of a method 200 for analyzing the health of a pancreas of an individual, which will generally be a human being. In some implementations, method 200 can be used to assess the individual's risk of developing a pancreatic ductal adenocarcinoma (PDAC), which is a type of cancerous tumor that forms in the ducts of the pancreas. As used herein the terms “a PDAC” and “the PDAC” generally refer to the actual tumor that has developed or may develop in the pancreas. In some instances, however, “PDAC” may also be used to refer to the general state of an individual who has develop a PDAC in their pancreas. Step 210 of the method 200 includes receiving a CT image of the individual's pancreas. In general, the CT image shows the individual's pancreas at a time prior to any visible evidence that a PDAC has developed in the pancreas and/or any diagnosis of a PDAC by a healthcare provider.

Step 220 of the method 210 includes analyzing the CT image to determine the value of each of one or more radiomic features of the CT image. The one or more radiomic features can include, for example, (i) a first set of one or more features associated with an intensity of one or more pixels of the CT image, (ii) a second set of one or more features associated with one or more shapes formed by the one or more pixels of the CT image, (iii) a third set of one or more features associated with a variation in intensity of the one or more pixels of the CT image, (iv) a fourth set of one or more features associated with one or more transformations applied to the one or more pixels of the CT image, (v) a fifth set of one or more features associated with one or more filters applied to the one or more pixels of the CT image, (vi) any combination of (i)-(v). In some implementations, the one or more radiomic features of the CT image includes a long-run low grey-level emphasis, a short-run low grey-level emphasis, a gaussian left polar, an inverse gaussian left polar, an inverse cluster shade, an inverse cluster prominence, an inverse cluster tendency, or any combination thereof. In general, any number of radiomic features can be analyzed.

Step 230 includes determining a PDAC risk factor for the individual's pancreas based at least in part on the value of at least some of the radiomic features. In some implementations, the PDAC risk factor includes a probability that a PDAC will develop in the pancreas within a certain time frame. In some implementations, the PDAC risk factor includes an indication of whether the pancreas has a high risk or a low risk of developing a PDAC in the future.

The pancreas is generally divided into a plurality of different regions. These regions can include a head region, a tail region, and a body region between the head region and the trail region. These regions and may in some cases also include a neck region positioned between the head region and the body region. In some implementations, the PDAC risk factor is determined for each region and/or is based on radiomic features for each region. For example, step 220 can include segmenting the pancreas into different regions and determining the values of the radiomic features within each region. Step 230 can then include analyzing the values of the different region's radiomic features separately to determine the PDAC risk factor.

In some implementations, determining the PDAC risk factor may include determining multiple PDAC risk factors for the pancreas. For example, the tail region and body region PDAC risk factors can indicate that the tail region and body region both have a low risk of developing a PDAC, while the head region PDAC risk factor can indicate that the head region has a high risk of developing a PDAC. In other implementations, only a single PDAC risk factor is determined, but it is based on the individual analysis of the different regions. For example, in some cases the PDAC risk factor indicates that the pancreas has a low risk of developing a PDAC if the risk of each individual region developing a PDAC is low, and indicates that the pancreas has a high risk of developing a PDAC if the risk of any single region developing a PDAC is high. In some implementations, if the PDAC risk factor indicates that the pancreas has a high risk of developing a PDAC, the PDAC risk factor may also indicate which region or regions individually have a high risk. In some implementations, the PDAC risk factor can include an indication of which of the regions will likely have the majority of a PDAC if the pancreas develops a PDAC in the future.

In some implementations, a machine learning model can be used to implement all or part of method 200. For example, in some implementations, analyzing the CT image at step 220 and determining the PDAC risk factor at step 230 can include inputting the CT image into a machine learning model which is trained to analyze the CT image and generate the PDAC risk factor as its output. The machine learning model can be trained to analyze the CT image to determine the value of the radiomic features (across the whole pancreas and/or for each region within the pancreas), and to determine the PDAC risk factor based on the radiomic feature values. Thus, in these implementations, the machine learning model is trained to receive a CT image and to output the PDAC risk factor. The machine learning model in these implementations may also output the radiomic feature values as well.

In some implementations, the machine learning model is used only to implement step 230. Thus, determining the radiomic feature values at step 220 can be done by a user, technician, healthcare provider, etc. (for example using non-machine learning-based image processing techniques). The radiomic feature values can then be input into the machine learning model, which then outputs the PDAC risk factor. Thus, in these implementations, the machine learning model is trained to receive radiomic features values and to output the PDAC risk factor.

In some implementations, the machine learning model analyzes the individual regions of the pancreas. Thus, in implementations where the machine learning model receives a CT image, the machine learning model can segment the pancreas in the CT image into the different regions, determine the radiomic feature values within each region, and determine the PDAC risk factor based on these radiomic feature values (e.g., a single PDAC risk factor based on the analysis of multiple regions, separate PDAC risk factors for each region, etc.). In implementations where the machine learning model receives the radiomic feature values, the machine learning model can analyze the radiomic feature values of the different regions and determine the PDAC risk factor based on these radiomic feature values (e.g., a single PDAC risk factor based on the analysis of multiple regions, separate PDAC risk factors for each region, etc.).

In some implementations, the machine learning model is a naive Bayes classifier that is trained with a training data set that includes a plurality of training CT images and/or radiomic feature values obtained from training CT images. The training CT images can be CT images that show a pancreas that is known to have later developed a PDAC, and can also be referred to as pre-diagnostic CT images (e.g., the CT images were generated prior to any diagnosis of a PDAC by a healthcare provider). The CT images themselves can be used to train the naive Bayes classifier, or radiomic feature values obtained from the CT images can be used to train the naive Bayes classifier. In some implementations, the naive Bayes classifier is trained using recursive feature elimination to identify a subset of radiomic features that will actually be used by the trained naive Bayes classifier. For example, a large number of radiomic features can initially be used to train the naive Bayes classifier, and the use of recursive feature elimination can eliminate radiomic features that are less important to determining the PDAC risk factor in order to form the subset of radiomic features.

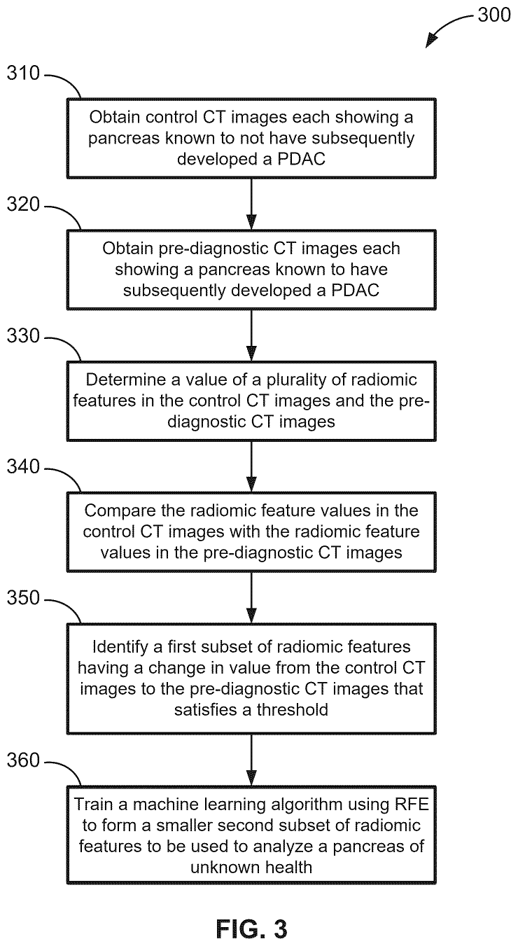

FIG. 3 shows a flow chart of a method 300 for training a machine learning model to analyze pancreas health. The machine learning model that is trained using the method 30 can be used, for example, to implement all or parts of method 200. Step 310 includes obtaining a plurality of control CT images, where each control CT image shows a respective pancreas that is known to have not subsequently developed a PDAC within a certain time period after acquisition of the control CT image. In some implementations, each pancreas shown in a control CT image is known to have not developed a PDAC within 1 year, 1.5 years, 2 years, 2.5 years, or 3 years following the acquisition of the control CT image.

Step 320 of method 300 includes obtaining pre-diagnostic CT images, where each pre-diagnostic CT image shows a respective pancreas that is known to have subsequently developed a PDAC within a certain time period after acquisition of the pre-diagnostic CT image. In some implementations, each pancreas shown in a pre-diagnostic CT image is known to have developed a PDAC within 1 year, 1.5 years, 2 years, 2.5 years, or 3 years following the acquisition of the control CT image. In some implementations, the time period of the pre-diagnostic CT images matches the time period for the control CT images. For example, in some of these implementations, of all of the control CT images show a pancreas known to not have developed a PDAC within the 3 years following acquisition of the control CT image, each pre-diagnostic CT image shows a pancreas known to have developed a PDAC within the 3 years following acquisition of the pre-diagnostic CT image.

Step 330 includes determining the value of each of a plurality of radiomic features in both the control CT images and the pre-diagnostic CT images. Generally, the same set of radiomic features is used for both the control CT image and the pre-diagnostic CT images. The plurality of radiomic features can include, for example, (i) a first set of one or more features associated with an intensity of one or more pixels of the CT image, (ii) a second set of one or more features associated with one or more shapes formed by the one or more pixels of the CT image, (iii) a third set of one or more features associated with a variation in intensity of the one or more pixels of the CT image, (iv) a fourth set of one or more features associated with one or more transformations applied to the one or more pixels of the CT image, (v) a fifth set of one or more features associated with one or more filters applied to the one or more pixels of the CT image, (vi) any combination of (i)-(v).

Step 340 includes comparing the values of the plurality of radiomic features in the control CT images with the values of the plurality of radiomic features in the pre-diagnostic CT images. Step 350 includes identifying a first subset of radiomic features of interest from the larger plurality of radiomic features. The first subset of radiomic features of interest will be used for further training. In some implementations, the radiomic features in the first subset of radiomic features are those whose change in value from the control CT images (showing healthy pancreases) to the pre-diagnostic CT images (showing pancreases that will develop a PDAC in the future) satisfy a predetermined threshold. In some implementations, the threshold is a threshold p-value for each radiomic feature after performing a statistical t-test on the compared radiomic feature values. For example, the threshold p-value could be 0.05. Any radiomic feature whose p-value is at or below this threshold p-value after performing the statistical t-test on the compared radiomic feature values can be placed into the first subset of radiomic features.

Thus, the radiomic features within the first subset of radiomic features can be radiomic features that show a significant change from a healthy pancreas (in the control CT images) to a pancreas that will develop or will likely develop a PDAC in the future (from the pre-diagnostic CT images), which are thus more indicative of whether a pancreas of unknown health will develop or will likely develop a PDAC in the future. Radiomic features that do not change much between a healthy pancreas and an unhealthy pancreas can be discarded, as they will not be of much use in discriminating between an unknown pancreas that is healthy and an unknown pancreas that will develop or will likely develop a PDAC in the future.

Step 360 includes training the machine learning model (which may include a naive Bayes classifier or other models) with the values of the first subset of radiomic features of interest in conjunction with recursive feature elimination (RFE). The use of RFE eliminates at least one radiomic feature from the first subset of radiomic features to form a second subset of radiomic features. This second subset of radiomic features includes fewer radiomic features than the first subset, and is the set of radiomic features that the trained machine learning model will use to analyze a CT image/radiomic features values corresponding to a pancreas of unknown health. In some implementations, the radiomic features in the second subset of radiomic features includes a long-run low grey-level emphasis, a short-run low grey-level emphasis, a gaussian left polar, an inverse gaussian left polar, an inverse cluster shade, an inverse cluster prominence, an inverse cluster tendency, or any combination thereof. In some implementations, the machine learning model determines the values of the second subset of radiomic feature when analyzing a CT image of a pancreas of unknown health, and then, as trained, uses those values to determine the PDAC risk factor. In other implementations, the values of the second subset of radiomic features are first obtained and then input into the machine learning model, which then, as trained, uses those values to determine the PDAC risk factor. In either implementation, once the machine learning model is trained, it can be used to analyze the health of an unknown pancreas in a CT image, for example using method 200.

In some implementations, the PDAC risk factor that the machine learning model is trained to output is based on the pancreas having multiple regions. Similar to method 200, the machine learning model can be trained to output multiple PDAC risk factors that each correspond to different regions of the pancreas, or a single PDAC risk factor that is based on the individual analysis of the different regions. In these implementations, the machine learning model can be trained to output a PDAC risk factor indicating a high risk if at least one of the regions of the pancreas has a high risk, and to output a PDAC risk factor indicating a low risk if all of the regions of the pancreas have a high risk.

In some implementations, various steps of method 300 are broken down into region-by-region actions. For example, determining the value of the radiomic features in the pre-diagnostic CT images in step 330 can include dividing each of the pre-diagnostic CT images into a plurality of regions that corresponds to the regions of the pancreas, and then determining the value of the radiomic features within each of these regions. Thus, the pancreas in the pre-diagnostic CT images is divided in the same manner as the actual pancreas to allow for a more granular analysis of the pancreas.

In order to compare the radiomic feature values between the control CT images and the pre-diagnostic CT images in implementations where the pre-diagnostic CT images are divided into regions, method 300 can further include dividing the control CT images into a plurality of regions that correspond to the pancreas of each control CT image. Thus, like the pre-diagnostic CT images, the pancreas in the control CT images is divided in the same manner as the actual pancreas.

Once the control CT images are divided into the regions, comparing the radiomic feature values between the control CT images and the pre-diagnostic CT images in step 340 (to identify the first subset of radiomic features of interest) can include comparing the radiomic feature values only between the same regions. For example, the pancreas (real and shown in the CT images) may be divided into a head region, a body region, and a tail region. The radiomic feature values in the head region of the control CT images can be compared to the radiomic feature values in the head region of the pre-diagnostic CT images, the radiomic feature values in the body region of the control CT images can be compared to the radiomic feature values in the body region of the pre-diagnostic CT images, and the radiomic feature values in the tail region of the control CT images can be compared to the radiomic feature values in the tail region of the pre-diagnostic CT images. Based on these comparisons, the first subset of radiomic features of interest can be identified. This region-by-region analysis can be use if there are, for example, certain radiomic features that are significantly different between a healthy pancreas and an unhealthy pancreas in one region, but not in another region. Thus, comparing the radiomic feature values on a region-by-region basis allows for more important radiomic features to be selected for the first subset of radiomic features.

In some implementations, the analysis can be performed at an even more granular level. In general, the different regions of a pre-diagnostic CT image may not all have the same risk of developing a PDAC in the future, even if the pancreas in that pre-diagnostic CT image is known to have developed a PDAC in the future. Thus, comparing radiomic features in one region of the control CT image to the radiomic features in that same region of a pre-diagnostic CT image may not be provide any useful information about whether those radiomic features are significant, if the PDAC that later developed in the pancreas of the pre-diagnostic CT image did not develop in that region. Thus, in some implementations, the regions of the pre-diagnostic CT images can be marked as high-risk or low-risk. The radiomic features from a respective region in the control CT images (which are known to have not later developed a PDAC and thus are low-risk) can then be compared to radiomic features from the same respective region in the pre-diagnostic CT images, but only from pre-diagnostic CT images where that region was high-risk and is known to have later developed a PDAC.

In these implementations, method 300 can further include obtaining diagnostic CT images that are used to mark the regions of the pre-diagnostic CT images as high-risk or low-risk. Each of the diagnostic CT images will correspond to one of the pre-diagnostic CT images, and will show the pancreas after it has developed the PDAC. Thus, the pre-diagnostic CT images and the diagnostic CT images can be formed in pairs, where each pair was acquired from the same patient. Each diagnostic CT image can then be divided into the same plurality of regions as its corresponding pre-diagnostic CT image. The individual regions of each diagnostic CT image can then be compared to the individual regions of the corresponding pre-diagnostic CT image, so that each region of each corresponding pre-diagnostic CT image can be marked as high-risk or low-risk.

In some implementations, a respective region of a pre-diagnostic CT image is marked as high-risk of at least a portion of the PDAC in the corresponding diagnostic CT image developed in the respective region. In some implementations, a respective region of a pre-diagnostic CT image is marked as high-risk of a majority of the PDAC in the corresponding diagnostic CT image developed in the respective region. In some implementations, a respective region of a pre-diagnostic CT image is marked as low-risk of no portion of the PDAC in the corresponding diagnostic CT image developed in the respective region. In some implementations, a respective region of a pre-diagnostic CT image is marked as low-risk if a minority of the PDAC in the corresponding diagnostic CT image developed in the respective region.

Other thresholds may also be used to mark regions in the pre-diagnostic CT images as high-risk or low-risk. For example, in some implementations, a respective region of a pre-diagnostic CT image is marked as high-risk if at least 25% of the PDAC in the corresponding diagnostic CT image developed in the respective region. In general, a respective region of a pre-diagnostic CT image can be marked as high-risk if at least n % of the PDAC in the corresponding diagnostic CT image developed in the respective region, and conversely, a respective region of a pre-diagnostic CT image can be marked as low-risk if at most 100-1% of the PDAC in the corresponding diagnostic CT image developed in the respective region. In these implementations, n % can be any suitable percentage, such as 20%, 25%, 30%, 35%, 40%, 45%, 50%, etc.

Once the regions in the pre-diagnostic CT images have been marked as high-risk or low-risk by comparing them to the regions in the corresponding diagnostic CT images, the radiomic feature values between the control CT images and the pre-diagnostic CT images can be compared. In some implementations, step 340 of method 300 includes comparing (i) the radiomic feature values in the respective regions of the control CT images to (ii) the radiomic feature values in the respective regions marked as high-risk in the pre-diagnostic CT images. In other implementations however, the low-risk regions of the pre-diagnostic CT images can also be used in the comparison. In these implementations, step 340 of method 300 includes comparing (i) the radiomic feature values in the respective regions of the control CT images and in the respective regions marked as low-risk in the pre-diagnostic CT images, to (ii) the radiomic feature values in the respective regions marked as high-risk in the pre-diagnostic CT images.

Once this risk-adjusted region-by-region comparison is done, the identification of the first subset of radiomic features can be performed at step 350. In these implementations, any feature whose change in value from (i) a respective region of the control CT images (and also the low-risk regions of the pre-diagnostic CT images in some implementations) to (ii) the respective region of the pre-diagnostic CT image satisfies the predetermined threshold can be included in the first subset of radiomic features. Thus, in some cases, a given radiomic feature measured in one region of the CT images/the pancreas can be included in the first subset of radiomic features while that same radiomic feature measured in a different region of the CT images/the pancreas is excluded from the first subset of radiomic features.

As discussed herein, the pancreas can have any number of different regions for purposes of training the machine learning model in method 300. In some implementations, the pancreas includes the head region, the body region, and the tail region, and each of the region-by-region steps discussed herein can be performed for each of these three regions. In other implementations, the pancreas additionally includes the neck region, and each of the region-by-region steps discussed herein can be performed for each of these four regions.

Disclosed herein is an example of the methods illustrated in FIG. 2 and FIG. 3.

### Introduction

Pancreatic Ductal Adenocarcinoma (PDAC) is a lethal cancer that accounts for more than 90% of pancreatic cancer incidences. At present, PDAC is the 4th key cause of cancer-related deaths, with a high expectancy to become the 2nd most by 2030, in both males and females. The American Cancer Society anticipates 62, 210 new incidences, and 49, 830 deaths, related to PDAC for the year 2022 in the US. The PDAC mostly remains subclinical in the initial stages but progresses rapidly once established. Resultantly, in more than 80% of the cases, cancer has already progressed to later stages by the time of diagnosis. The negative margin (RO) resection of the PDAC promises long-term survival which is only possible when the cancer is identified at its earliest stages. Treatment, whether surgical or non-surgical, initiated at later stages of the PDAC is associated with poor survival benefits. Although the current overall five-year survival rate of PDAC is barely 11.5%, recent research suggests that detecting PDAC in the earliest stage can increase the survival rate up to 50%.

Risk prediction of the PDAC assists in improving the chances of diagnosis at an early stage as follow-up surveillance of high-risk individuals on a regular basis would allow early intervention reducing the chance of missing the initial stages of the disease. However, since the conventional predictive biomarkers of PDAC lack specificity, risk prediction is challenging. Further, signs and symptoms of pancreatic cancer are either absent or are nonspecific as these are associated with several different diseases. Factors including the complex location and variability of the pancreas may underlie, in part, the difficulty with an early diagnosis with imaging.

The pancreas undergoes several morphological changes, both locally (e.g., subregional variations) and globally (alterations to the whole pancreas), during the development of PDAC. Empirical observations associate PDAC with several preconditioning disorders that usually lead to such morphological and textural changes in the pancreas. For example, complications including IPMN pancreatic tumors, distal parenchymal atrophy, and pancreatolithiasis (intraductal calculi) gradually increase the heterogeneity of the pancreatic tissue and can potentially be used as a noninvasive risk predictor. Other deformations may include shape and size variations in the pancreas that are consistently associated with ductal dilation and inflammation in the pancreas. However, studies reported that these alterations can be highly subtle and unique to each pancreatic subregion (the term pancreatic subregion and subregion are used interchangeably). For instance, tumor histology differs across pancreatic subregions (i.e., head, body, and tail) which causes spatial heterogeneity within the pancreas. Also, most of these microlevel variations are difficult to comprehend by visual assessment of abdominal imaging and require computer-based quantification.

AI is the primary choice to perform image-based extensive analysis of such minute alterations and identify potential risk predictors for disease. AI systems, as opposed to manual approaches, execute complex tasks without interruption and ensure highly accurate and precise outcomes. In the domain of automated processing and analysis of medical images, AI offers numerous techniques and tools to extract accurate measurements from different structures, identify nonlinear features, and evaluate tissue properties. For prediction modeling, radiomic analysis, and machine and deep learning are regarded as the most reliable and common AI approaches.

In this example, the precursory changes taking place across pancreatic subregions during cancer development were thoroughly examined and characterized the pancreas that is likely to develop PDAC. A rigorous radiomic analysis of morphological and textural features of three pancreatic subregions (head, body, tail) in the pre-diagnostic abdominal CT scans was performed to identify the features potentially predictive of cancer. Subsequently, a machine learning model was developed that performs risk prediction by automatically classifying the abdominal CT scans into the pre-diagnostic (pancreas has a high risk for cancer) and healthy control (pancreas has a low risk for cancer) groups and specifying the subregion of the pancreas that is expected to develop most part of the tumor than its neighboring subregions.

### Materials

### CT Imaging for PDAC Diagnosis

Of many imaging modalities, CT plays an important role in the screening for early detection of PDAC. During the initial evaluation of subjects with suspected PDAC, the abdominal CT examination is the common choice to seek primary and secondary signs of cancer. Two institutes, the Cedars-Sinai Medical Center (CSMC) and the Kaiser Permanente Southern California (KPSC) in Los Angeles, collaborated and provided eligible CT scans for analysis.

### Datasets for the Analysis

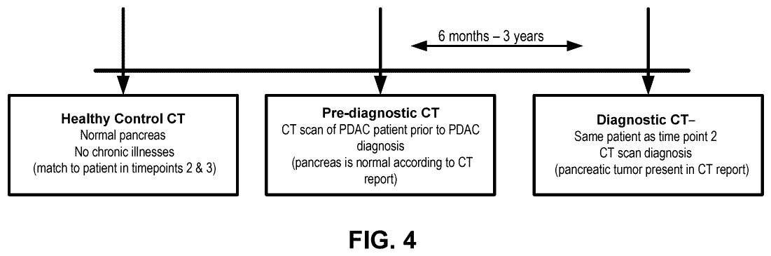

The data obtained for the study consisted of contrast enhanced abdominal CT scans from a diagnostic group, a pre-diagnostic group, and a healthy control group. The diagnostic scans belong to subjects with biopsy-confirmed PDAC and observable tumor on the CT scan. These patients do not have any history of pancreatic tumor resection. The pre-diagnostic scans were acquired for the same subject, as in the diagnostic class, 6 months to 3 years before their PDAC was diagnosed. No primary or secondary signs of PDAC were present at the time the pre-diagnostic scan was acquired. The healthy control scan was obtained for a different subject having healthy (“normal”) pancreas with no history of any pancreatic disorders. The gender and age of each subject in the healthy control class and the year their scan was acquired match those of exactly one unique subject in the pre-diagnostic class to reduce instrumental and morphologic differences, respectively. No subject in the healthy control class developed PDAC within the next 36 months of their scan. The data design is shown in FIG. 4.

The two institutes obtained 108 CT scans from 72 subjects and were divided into internal and external datasets. The former consists of 66 scans (22 from each of the three groups) and the latter consists of 42 scans (14 from each of the three groups) from 44 and 28 subjects at CSMC and KPSC respectively. Also, 58 scans (19 diagnostic, 17 pre-diagnostic, 22 healthy control) in the internal dataset and all 42 scans in the external dataset were venous phase images, whereas the rest of 8 scans in the internal dataset belong to multiple phases such as arterial, venous, and connecting phases. The external dataset was used for external validation of the proposed prediction model. Table 1 provides the split of both internal and external dataset.

TABLE 1

| | | | | | Number |
|---|---|---|---|---|---|
| | Healthy control | Pre-diagnostic | Diagnostic | Total | of |
| | scans | scans | scans | scans | subjects |
| Internal | 22 scans (20 | 22 scans (20 | 22 scans (20 | 66 | 44 |
| dataset | venous, 2 | venous, 2 | venous, 2 | | |
| | arterial) | arterial) | arterial) | | |
| External | 14 venous | 14 venous | 14 venous | 42 | 28 |
| dataset | scans | scans | scans | | |

### Data Reference Labeling and Preprocessing

For precise measurements of pancreatic features, accurate delineation of the pancreas and the subregions is a prerequisite. The anatomy of the pancreas is complex and requires considerable attention and skills during outlining the pancreas and its subregions. The general shape of the pancreas resembles a hockey stick (J-shaped) structure. On the axial view of an abdominal CT, the pancreas lies across the posterior abdomen. Anatomical subregions of the pancreas consist of the head, body, and tail that appear in the left-to-right order on the axial view of the CT. The head is the expanded medial part lying at the duodenum curve and is attached to the body subregion that connects to a tapered tail subregion. The anteroposterior diameter and the length of the pancreas usually lie between 1-3 cm and 12-15 cm with the head, body, and tail covering 40%, 33%, and 26% portion of the whole pancreas respectively.

Two experienced radiologists at CSMC manually outlined the boundary of the pancreas and three subregions in all 108 scans using the commercial software ITK-Snap. To avoid any prejudgment, findings or information attached to the scans from previous assessments were removed before labeling. A three-step labeling process was performed to ensure labeling consensus. In the first labeling phase, the two readers independently specified the boundary of the whole pancreas and subregions in all scans to limit the inter-reader variability, resulting in 85.4% labeling consistency. In the second phase, both readers were allowed to evaluate each other's labels and update their original labels which resulted in 97% labeling overlap. Lastly, the 3% labeling conflict in the updated label sets was discussed and resolved with mutual agreement of both graders.

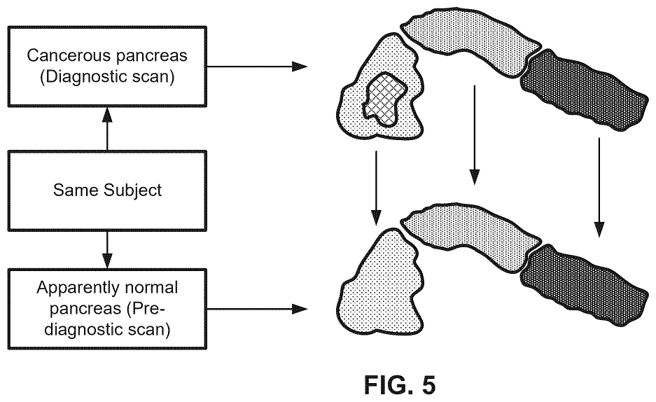

In each diagnostic scan, the readers also specified the subregion that contained the greatest amount of pancreatic tumor. This helped grade the subregions in the corresponding pre-diagnostic scans into high-risk and low-risk classes. For instance, if most parts of the tumor were observed in the head subregion of the pancreas in a diagnostic scan, then the head subregion in the corresponding pre-diagnostic scan was graded as a high-risk subregion, whereas the rest of the neighboring subregions in the same pre-diagnostic scan were graded as low-risk subregions, as given in FIG. 5. Multiple subregions were graded as high-risk in the same pre-diagnostic scan if the tumor was observed in more than one subregion in the corresponding diagnostic scan. Note that all subregions in the healthy control scans were graded as low-risk subregions. Moreover, from 132 subregions in 44 CT scans (22 healthy control, 22 pre-diagnostic) of the internal dataset, the grading identified a total of 66 and 44 low-risk subregions in healthy control and pre-diagnostic scans respectively, and 22 high-risk subregions in pre-diagnostic scans. For 84 subregions from 28 CT scans (14 healthy control, 14 pre-diagnostic) of the external dataset, the grading identified 42 and 28 low-risk subregions in healthy control and pre-diagnostic scans respectively, and 14 high-risk subregions in pre-diagnostic scans. Furthermore, the pancreas as a whole was graded as low-risk and high-risk in healthy control and pre-diagnostic groups respectively.

Each of the 108 scans has 16-bit depth and a slice resolution of 512 by 512 (along the x- and y-axis) and variable z-axis. No preprocessing was performed on any of the scans except the signal intensities in each scan were scaled between 0 and 1.

### Methods

Risk prediction modeling was carried out by thoroughly examining the morphology and the texture of the precancerous subregions to seek predictive features, followed by utilizing these features in a machine learning classifier to automatically characterize the pancreas and subregions into high-risk and low-risk classes for PDAC. The methodology is explained below.

### Radiomic Analysis of Pancreatic Subregions

A large amount of radiomic features were obtained from each of 194 subregions in 66 CT scans (22 healthy control, 22 pre-diagnostic, 22 diagnostic) of the internal dataset, e.g., three sets of features-one for each of the three groups, whereas each set consists of three subsets: one for each of three subregions. Each feature in the set expressed a unique quantifiable property of a subregion that provided information about the spatial relationship of neighboring voxels in predefined proximity. To calculate a numerical value for each feature, signal intensities of all 3D pixels specified within a volumetric subregion (all slices) of a scan were considered.

An important aspect of radiomic analysis is to consider the variations in a radiomic feature determined by the three parameters that include the kernel size, the angle, and the bin size. Different combinations of these parameters influence the entire analysis to a high extent. The kernel is the square convolution matrix that specifies the area (proximity) A surrounding a voxel x, for which the spatial relationships are calculated with its neighbors lying within area A. The angle specifies the directions when calculating associations of x with its neighbors within the area A. The bin size was the number used to discretize the continuous values of voxels in the CT image into their counter parts equal bins to avoid considering two pixels (having too-close signal intensities) any different. Each radiomic feature represented one of the major characteristics of a subregion that includes shape, size, texture, and signal intensity using a unique mathematical expression. Common types of radiomic features considered include first-order statistics (e.g., kurtosis, coefficient of variation, entropy) and higher-order statistics (e.g., contrast, homogeneity, coarseness). With different combinations of three parameters, around 4,000 radiomic features from each of 194 subregions were extracted by considering the whole subregion as a single region of interest.

Using the 132 subregions in 44 CT scans (22 healthy control, 22 pre-diagnostic) in the internal dataset, a pairwise feature comparison between the corresponding subregions (i.e., head-to-head, body-to-body, tail-to-tail) was performed to identify the features that were significantly different between high-risk and low-risk subregions. For example, the extracted features from all low-risk head subregions in the internal dataset were compared with the same set of features extracted from all high-risk head subregions in the internal dataset. About 3.5% of the extracted features showed significance (found potentially predictive) at a p-value of 0.05 in the statistical t-tests-supporting the core hypothesis about the presence of precancerous changes occurring locally within the subregions undergoing tumor development. Note that the only purpose of considering the features extracted from the 66 subregions in 22 CT diagnostic scans in the internal dataset during the analysis was to help to select the predictive features that are highly stable and do not become insignificant when pre-diagnostic and diagnostic scans are mixed.

### Risk Prediction of PDAC

The significant features (predictors) identified through the subregional analysis were used to perform automated risk prediction of PDAC by classifying the pancreas into either low-risk or high-risk categories. The criteria set to perform binary classification was to mark the pancreas as low-risk if none of its subregions was classified as high-risk, whereas the pancreas was marked as high-risk, if at least one of its subregions was classified as high-risk. A misclassification is counted if the classifier marks one or more subregions as high-risk in a healthy control scan, or if the classifier identifies a high-risk subregion as low-risk in the pre-diagnostic scan or vice versa.

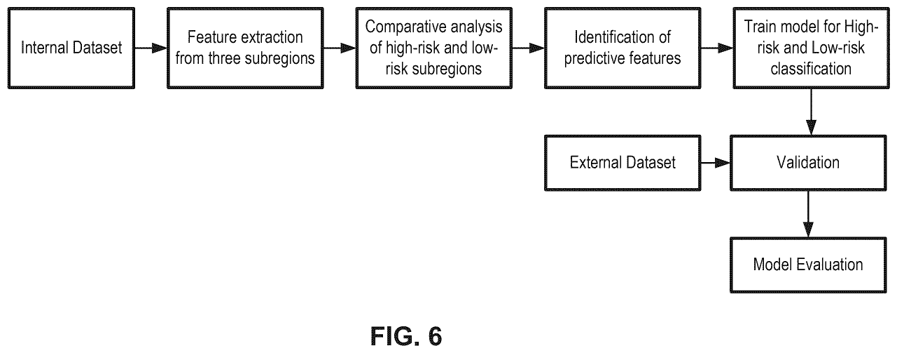

The naive Bayes (NB) model was trained for binary classification in conjunction with the Recursive Feature Elimination (RFE) method in which the RFE method eliminated the weak features using different combinations of identified predictors while maximizing the overall training accuracy based on the given classification criteria. Of note, the RFE was prespecified to select up to the seven best features to avoid overfitting the NB classifier. The NB-RFE identified seven features (long-run low grey-level emphasis, gaussian left polar, inverse gaussian left polar, inverse cluster shade, inverse cluster prominence, inverse cluster tendency, and short-run low grey-level emphasis) as the best predictors for the classifier to get the maximum classification accuracy during training the model on all the 44 CT scans (132 subregions) of the internal dataset. The external validation of the trained model was then performed using 24 CT scans (84 subregions) of the external dataset. An overview of the prediction process is provided in FIG. 6.

### Results

Model performance was evaluated in terms of classification accuracy, sensitivity, and specificity. The classification accuracy was calculated as the total number of correctly classified scans (both healthy control and pre-diagnostic) to the total number of scans input to the NB classifier. The sensitivity is the true positive rate which refers to the total number of correctly classified pre-diagnostic scans (high-risk pancreas) to the total number of pre-diagnostic scans input to the NB classifier. Whereas the specificity is the true negative rate which refers to the total number of correctly classified healthy control scans (low-risk pancreas) to the total number of healthy control scans input to the NB classifier.

The mean classification accuracy achieved on the training data (internal dataset) was 93% (41/44), i.e., the number of correctly classified scans to the total number of scans observed. The external validation of the classifier was performed using the 56 subregions in 28 scans (14 healthy control and 14 pre-diagnostic) in the external dataset. The validation achieved the mean classification accuracy of 89.3% (25/28), with the sensitivity and specificity reaching 86% and 93% respectively, as given in the confusion matrix Table 2.

TABLE 2

| | True Healthy | True Pre-diagnostic |
|---|---|---|
| Predicted Healthy | 13 | 2 |
| Predicted Pre-diagnostic | 1 | 12 |

The proposed model demonstrated improved accuracy by 3.3% compared to past efforts. Also, it was empirically observed that the inter-variability between the features extracted from corresponding low-risk subregions identified in healthy control and pre-diagnostic scans was significantly low at a p-value of 0.05. This supports the primary hypothesis that the precancerous changes predominately occur locally and are specific to the subregion within which the tumor is likely developing. Also, the 95% confidence interval (CI) achieved in the current study is 78-100, showing modest improvement on the lower bound of the CI obtained in the previous study (i.e., 73-99). Further improvement in the current CI was possible if the model training was not enforced to use a fixed limited number of predictors to avoid model overfitting.

Moreover, the radiomic analysis infers that it is essentially the texture of the pancreas that changes locally and appears abnormal on a CT scan during cancer development. These textural changes are the possible indication of the stage the underlying healthy cells are transitioning into tumor cells (e.g., the tumorous region turns more hypointense than the nontumorous peripheral region on a CT image). Furthermore, the shape of the whole pancreas (in healthy and pre-diagnostic scans) and subregions (belong to high-risk and low-risk classes) was observed indifferent, partly because the shape of the pancreas is highly irregular in general. However, the size of the high-risk subregions was observed slightly higher than their corresponding low-risk subregions, though not significantly different to be considered a stable predictor.

### Discussion

### Clinical Significance of PDAC Prediction Using CT Imaging

The Centers for Disease Control and Prevention reports that 7 million patients with abdominal pain visit to ER in the US each year. These patients undergo CT examinations as per the standard care protocol. The initial evaluation of these scans assists clinicians to identify the underlying cause of abdominal pain. Though the scans of majority of these patients do not present any signs of cancer at this stage, some ultimately develop PDAC in coming years. These pre-diagnostic scans, even with no prominent signs of cancer, are clinically useful as these might contain significant morphological signatures of early biological adaptations associated with cancer. In this example, the quantitative difference of the CT-based features between pre-diagnostic and healthy control scans was examined, which allows for quantitative analysis of the subregional changes that occurred in the precancerous or pre-symptomatic pancreas and helps reduce limitations of low prevalence and low cancer yield in prospective studies as half of the subjects have cancer.

The unique data structure designed for this study is the foundation of the proposed prediction model as it allowed examining precancerous changes retrospectively. Although the overall prevalence of PDAC is significantly low, the percentage of enrolled subjects who were at the preclinical stage was set to 50% to reduce the risk of class imbalance during model development. Also, most of the literature considers that the duration of 6-36 months between the pre-diagnostic and diagnostic scan is a reasonable window to seek early signs.

Also, most of the scans used for mode training and testing are portal venous phase. It is because tumors slowly uptake contrast whereas the venous phase provides the optimal view of the tumor edges and is thus considered the most valuable phase for PDAC diagnosis. Also, viewing of the vasculature passing across or alongside the pancreas is optimized in this phase. Changes occurring to the vasculature during PDAC development can be quantified and used as potential predictors. Nevertheless, other phases also provide valuable information during PDAC screening and treatment. For example, the arterial phase provides a unique value when seeking lesions or during surgical treatment of PDAC when the arteries are encased or distorted by the pancreatic tumor. Thus, including multiphase scans in the model training helped identify highly stable predictors to ensure the model is sufficiently robust.

In accordance with the evidence provided, the proposed research work assures the appropriate blend of imaging type, feature analysis, and modeling techniques to address the challenges of prediction and elevate the chances of cancer diagnosis in the earliest stage. It is the first automated system developed that predicts the PDAC by identifying early signs through analyzing the precancerous irregularities occurring within pancreatic subregions using CT scans. The proposed model not only demonstrated improved prediction accuracy to existing models but also enabled the system to identify subregions that are at higher risk of developing tumors.

### Significance of the Subregional Analysis

Several studies suggest that tumor development differs across pancreatic subregions (Head: H, Body: B, Tail: T) in terms of histology, presentation, and symptoms. For instance, tumors in the head are mostly non-squamous, whereas the body and tail tumors are usually squamous. This results in spatial heterogeneity and various discrepancies across the pancreatic sub-regions; such as tumor presentation (e.g., head tumors are usually well-differentiated and less aggressive than those in body/tail), related symptoms (head tumors: unexplained weight loss, body tumors: pain in the upper abdomen, tail tumors: pain in the lower abdomen), sensitivity to drugs (head tumors are highly responsive to Gemcitabine regimen and less responsive to Fluorouracil regimen, whereas the body and tail tumors are vice-versa), and the different rates of incidence (H: 71%, B: 13%, T: 16%), metastasis (H: 42%, B: 68%, T: 84%), %), 2-year survival (H: 44%, B: 27%, T: 27%), and resection (H: 17%, B: 4%, T: 7%).

This study examined the subregional changes in the precancerous pancreas and enabled automated identification of subregions undergoing tumor development. Knowledge of the location of likely tumor will not only alert clinicians/radiologists to pay attention to certain regions of the pancreas to avoid misdetection of PDAC at an early stage but also enhance the overall management of PDAC by helping determine more appropriate and effective treatment, improving forecasting of the treatment outcome, planning better resection, and ultimately increasing the overall survival rate.

### Conclusion

The current study presented the findings of the AI analysis of precancerous changes that occurred across three subregions of the pancreas using pre-diagnostic abdominal CT scans. The study concluded that the pancreas adopts textural changes during PDAC development, predominantly within the subregion undergoing tumor development, potentially regarded as a high-risk subregion. A first model was built that performed risk quantification of PDAC using the identified textural changes as potential predictors and characterized the pancreas into high-risk and low-risk for PDAC classes. The model also specified the subregion that is likely to develop the tumor, which can potentially assist in improving early diagnosis, treatment planning, forecasting treatment outcome, and overall disease management. The proposed model demonstrates a 3.3% improved prediction when compared with the existing prediction model that considers the global changes occurring in the whole pancreas during PDAC development. The results of this preliminary study are promising and encouraging to further validate the model on a large dataset.

### ALTERNATIVE IMPLEMENTATIONS

Alternative Implementation 1. A method for analyzing health of a pancreas of an individual, the method comprising: receiving a computed tomography (CT) image of a pancreas of the individual; analyzing the CT image to determine a value of each of one or more radiomic features of the CT image; and based on the value of each of the one or more radiomic features of the CT image, determining a pancreatic ductal adenocarcinoma (PDAC) risk factor for the pancreas of the individual.

Alternative Implementation 2. The method of Alternative Implementation 1, wherein the PDAC risk factor includes a probability of the pancreas of the individual developing a PDAC.

Alternative Implementation 3. The method of Alternative Implementation 1 or Alternative Implementation 2, wherein the PDAC risk factor includes an indication of whether a risk of the pancreas of the individual developing a PDAC is low or high.

Alternative Implementation 4. The method of Alternative Implementation 3, wherein the pancreas of the individual includes a plurality of regions, and wherein the PDAC risk factor includes an indication of whether a risk of each of the plurality of regions developing a PDAC is low or high.

Alternative Implementation 5. The method of Alternative Implementation 3 or Alternative Implementation 4, wherein the pancreas of the individual includes a plurality of regions, the risk of the pancreas developing a PDAC being low if a risk of each of the plurality of regions developing a PDAC is low, the risk of the pancreas developing a PDAC being high if the risk of at least one of the plurality of regions developing a PDAC is high.

Alternative Implementation 6. The method of any one of Alternative Implementations 1 to 5, wherein the pancreas of the individual includes a plurality of regions, and wherein the PDAC risk factor includes an indication of which of the plurality of regions has a highest risk of developing a PDAC.

Alternative Implementation 7. The method of any one of Alternative Implementations 1 to 6, wherein the pancreas of the individual includes a plurality of regions, and wherein the PDAC risk factor includes an indication of which of the plurality of regions will contain a majority of a PDAC if the PDAC develops in the pancreas in the future.

Alternative Implementation 8. The method of any one of Alternative Implementations 1 to 7, wherein the one or more radiomic features of the CT image include (i) a first set of one or more features associated with an intensity of one or more pixels of the CT image, (ii) a second set of one or more features associated with one or more shapes formed by the one or more pixels of the CT image, (iii) a third set of one or more features associated with a variation in intensity of the one or more pixels of the CT image, (iv) a fourth set of one or more features associated with one or more transformations applied to the one or more pixels of the CT image, (v) a fifth set of one or more features associated with one or more filters applied to the one or more pixels of the CT image, (vi) any combination of (i)-(v).

Alternative Implementation 9. The method of any one of Alternative Implementations 1 to 8, wherein the one or more radiomic features of the CT image includes a long-run low grey-level emphasis, a short-run low grey-level emphasis, a gaussian left polar, an inverse gaussian left polar, an inverse cluster shade, an inverse cluster prominence, an inverse cluster tendency, or any combination thereof.

Alternative Implementation 10. The method of any one of Alternative Implementations 1 to 9, wherein analyzing the CT image and determining the PDAC risk factor includes: inputting the CT image into a machine learning model; and receiving the PDAC risk factor as an output of the trained machine learning model.

Alternative Implementation 11. The method of Alternative Implementation 10, wherein the machine learning model is trained to analyze the CT image to determine the value of each of the one or more radiomic features of the CT image and determine the PDAC risk factor based on the value of each of the one or more radiomic features of the CT image.

Alternative Implementation 12. The method of Alternative Implementation 10 or Alternative Implementation 11, wherein the machine learning model is a naive Bayes classifier.

Alternative Implementation 13. The method of Alternative Implementation 12, wherein the naive Bayes classifier is trained in conjunction with recursive feature elimination.

Alternative Implementation 14. The method of Alternative Implementation 12, wherein the naive Bayes classifier is trained with a training data set that includes a set of feature values obtained from a plurality of training CT images, the set of feature values including values of each of a plurality of features of each of the plurality of training CT images.

Alternative Implementation 15. The method of Alternative Implementation 14, wherein the naive Bayes classifier is trained using recursive feature elimination to identify a subset of the plurality of features to be used to determine the PDAC risk factor, the subset of the plurality of features identified during the training forming the one or more radiomic features used to determine the PDAC risk factor for the pancreas of the individual.

Alternative Implementation 16. A method of training a machine learning model to analyze pancreas health, the method comprising: obtaining a plurality of pre-diagnostic computed tomography (CT) images, each of the plurality of pre-diagnostic CT images showing a respective pancreas known to have subsequently developed a pancreatic ductal adenocarcinoma (PDAC); determining a value of each of a plurality of radiomic features in each of the plurality of pre-diagnostic CT images; and training the machine learning model to output a PDAC risk factor using the value of at least some of the plurality of radiomic features in each of the plurality of pre-diagnostic CT images, the PDAC risk factor being indication of whether a risk of a PDAC developing in a pancreas of an individual is high or low.

Alternative Implementation 17. The method of Alternative Implementation 16, further comprising: obtaining a plurality of control CT images, each of the plurality of control CT images showing a respective pancreas known to have not subsequently developed a PDAC; determining a value of each of the plurality of radiomic features in each of the plurality of control CT images; comparing the values of the radiomic features in the pre-diagnostic CT images with the values of the radiomic features in the control CT images; and based on the comparing, identifying a first subset of radiomic features of interest from the plurality of radiomic features, wherein the at least some of the plurality of radiomic features used to train the machine learning model includes only the radiomic features in the first subset.

Alternative Implementation 18. The method of Alternative Implementation 17, wherein training the machine learning model includes using recursive feature elimination to eliminate at least one radiomic feature from the first subset of radiomic features to form a second subset of radiomic features, the second subset of radiomic features including fewer radiomic features than the first subset of radiomic features.

Alternative Implementation 19. The method of Alternative Implementation 18, wherein the radiomic features in the second subset of radiomic features includes a long-run low grey-level emphasis, a short-run low grey-level emphasis, a gaussian left polar, an inverse gaussian left polar, an inverse cluster shade, an inverse cluster prominence, an inverse cluster tendency, or any combination thereof.

Alternative Implementation 20. The method of any one of Alternative Implementations 17 to 19, wherein the pancreas of the individual and the respective pancreas of each of the plurality of pre-diagnostic CT images includes a plurality of regions, and wherein training the machine learning model includes training the machine learning model to (i) output the PDAC risk factor indicating that the risk of the pancreas of the individual developing a PDAC is high if the risk of at least one of the plurality of regions of the pancreas of the individual developing a PDAC is high, and (ii) output the PDAC risk factor indicating that the risk of the pancreas of the individual developing a PDAC is low if the risk of each of the plurality of regions of the pancreas of the individual developing a PDAC is low.

Alternative Implementation 21. The method of Alternative Implementation 20, wherein determining the value of each of the plurality of radiomic features in each of the plurality of pre-diagnostic CT images includes: dividing each of the pre-diagnostic CT images into a plurality of regions corresponding to the plurality of regions of the respective pancreas of each of the plurality of pre-diagnostic CT images; and determining the value of each of the plurality of radiomic features in each of the plurality of regions of each of the plurality of pre-diagnostic CT images.

Alternative Implementation 22. The method of Alternative Implementation 21, further comprising: obtaining a plurality of diagnostic CT images, each of the plurality of diagnostic CT images corresponding to a respective one of the plurality of pre-diagnostic CT images and showing the respective pancreas after developing the PDAC; dividing each of the plurality of diagnostic CT images into a plurality of regions corresponding to the plurality of regions of the corresponding one of the plurality of pre-diagnostic CT images; and marking each region of each pre-diagnostic CT image as high-risk or low-risk based on comparing each region of each diagnostic CT image to the corresponding region of the corresponding pre-diagnostic CT image.

Alternative Implementation 23. The method of Alternative Implementation 22, wherein each respective region of each pre-diagnostic CT image is marked as high-risk if at least a portion of the PDAC in the corresponding diagnostic CT image is developed in the respective region.

Alternative Implementation 24. The method of Alternative Implementation 22 or Alternative Implementation 23, wherein each respective region of each pre-diagnostic CT image is marked as high-risk if a majority of the PDAC in the corresponding diagnostic CT image is developed in the respective region.

Alternative Implementation 25. The method of any one of Alternative Implementations 22 to 24, wherein each respective region of each pre-diagnostic CT image is marked as low-risk if no portion of the PDAC in the corresponding diagnostic CT image is developed in the respective region.

Alternative Implementation 26. The method of any one of Alternative Implementations 22 to 25, wherein each respective region of each pre-diagnostic CT image is marked as low-risk if a minority of the PDAC in the corresponding diagnostic CT image is developed in the respective region.

Alternative Implementation 27. The method of any one of Alternative Implementations 22 to 26, further comprising dividing each of the control CT images into a plurality of regions corresponding to the plurality of regions of the respective pancreas of each of the plurality of control CT images.

Alternative Implementation 28. The method of Alternative Implementation 27, wherein comparing the values of the radiomic features in the pre-diagnostic CT images to the values of the radiomic features in the control CT images includes, for each respective region of the plurality of regions, comparing (i) the values of the radiomic features in the respective regions of the control CT images and the respective regions marked as low-risk in the pre-diagnostic CT images to (ii) the values of the radiomic features in the respective regions marked as high-risk in the pre-diagnostic CT images.

Alternative Implementation 29. The method of Alternative Implementation 28, wherein each radiomic feature in the first subset of radiomic features of interest has a change in value from (i) the respective regions of the control CT images and the respective regions marked as low-risk in the pre-diagnostic CT images to (ii) the respective regions of the pre-diagnostic CT images, where the change in value satisfies a predetermined threshold.

Alternative Implementation 30. The method of any one of Alternative Implementations 22 to 29, wherein the plurality of regions includes a head region, a body region, and a tail region.

Alternative Implementation 31. The method of Alternative Implementation 30, wherein comparing the values of the radiomic features in the pre-diagnostic CT images to the values of the radiomic features in the control CT images includes: for the head region, comparing (i) the values of the radiomic features in the head regions of the control CT images and the head regions marked as low-risk in the pre-diagnostic CT images with (ii) the values of the radiomic features in the head regions marked as high-risk in the pre-diagnostic CT images; for the body region, comparing (i) the values of the radiomic features in the body regions of the control CT images and the body regions marked as low-risk in the pre-diagnostic CT images with (ii) the values of the radiomic features in the body regions marked as high-risk in the pre-diagnostic CT images; and for the tail region, comparing (i) the values of the radiomic features in the tail regions of the control CT images and the tail regions marked as low-risk in the pre-diagnostic CT images with (ii) the values of the radiomic features in the tail regions marked as high-risk in the pre-diagnostic CT images.

Alternative Implementation 32. The method of Alternative Implementation 31, wherein: for the head region, each radiomic feature in the first subset of radiomic features of interest has a change in value from (i) the head regions of the control CT images and the head regions marked as low-risk in the pre-diagnostic CT images to (ii) the head regions of the pre-diagnostic CT images, where the change in value satisfies a predetermined threshold; for the body region, each radiomic feature in the first subset of radiomic features of interest has a change in value from (i) the body regions of the control CT images and the body regions marked as low-risk in the pre-diagnostic CT images to (ii) the body regions of the pre-diagnostic CT images, where the change in value satisfies the predetermined threshold; and for the tail region, each radiomic feature in the first subset of radiomic features of interest has a change in value from (i) the tail regions of the control CT images and the tail regions marked as low-risk in the pre-diagnostic CT images to (ii) the tail regions of the pre-diagnostic CT images, where the change in value satisfies the predetermined threshold.

Alternative Implementation 33. A method of training a machine learning model to analyze pancreas health, the method comprising: obtaining a plurality of control computed tomography (CT) images, each of the plurality of control CT images showing a respective pancreas known to have not subsequently developed a PDAC; obtaining a plurality of pre-diagnostic CT images, each of the plurality of pre-diagnostic CT images showing a respective pancreas known to have subsequently developed a pancreatic ductal adenocarcinoma (PDAC); determining a value of each of a plurality of radiomic features in each of the plurality of pre-diagnostic CT images and in each of the plurality of control CT images; comparing the values of the radiomic features in the control CT images with the values of the radiomic features in the pre-diagnostic CT images; based on the comparing, identifying a first subset of radiomic features of interest from the plurality of radiomic features, each radiomic feature in the first subset of radiomic features of interest having a change in value from the control CT images to at least a portion of the pre-diagnostic CT images that satisfies a predetermined threshold; and training the machine learning model in conjunction with recursive feature elimination to eliminate at least one radiomic feature from the first subset of radiomic features to form a second subset of radiomic features, the second subset of radiomic features including fewer radiomic features than the first subset of radiomic features, the trained machine learning model outputting a PDAC risk factor based on only the values of the radiomic features in the second subset of radiomic features.

Alternative Implementation 34. A system comprising a control system configured to implement the method of any one of Alternative Implementations 1 to 33.

Alternative Implementation 35. A computer program product comprising instructions which, when executed by a computer, cause the computer to carry out the method of any one of Alternative Implementations 1 to 33.

Alternative Implementation 36. The computer program product of Alternative Implementation 35, wherein the computer program product is a non-transitory computer readable medium.

Alternative Implementation 37. A system comprising: a memory device having stored thereon machine-readable instructions; and a control system including one or more processors configured to execute the machine-readable instructions to: receive a computed tomography (CT) image of a pancreas of the individual; analyze the CT image to determine a value of each of one or more radiomic features of the CT image; and based on the value of each of the one or more radiomic features of the CT image, determine a pancreatic ductal adenocarcinoma (PDAC) risk factor for the pancreas of the individual.

Alternative Implementation 38. A system comprising: a memory device having stored thereon machine-readable instructions; and a control system including one or more processors configured to execute the machine-readable instructions to: obtain a plurality of pre-diagnostic computed tomography (CT) images, each of the plurality of pre-diagnostic CT images showing a respective pancreas known to have subsequently developed a pancreatic ductal adenocarcinoma (PDAC); determine a value of each of a plurality of radiomic features in each of the plurality of pre-diagnostic CT images; and train the machine learning model to output a PDAC risk factor using the value of at least some of the plurality of radiomic features in each of the plurality of pre-diagnostic CT images, the PDAC risk factor being indication of whether a risk of a PDAC developing in a pancreas of an individual is high or low.

Alternative Implementation 39. A system comprising: a memory device having stored thereon machine-readable instructions; and a control system including one or more processors configured to execute the machine-readable instructions to: obtain a plurality of pre-diagnostic computed tomography (CT) images, each of the plurality of pre-diagnostic CT images showing a respective pancreas known to have subsequently developed a pancreatic ductal adenocarcinoma (PDAC); obtain a plurality of control CT images, each of the plurality of control CT images showing a respective pancreas known to have not subsequently developed a PDAC; determine a value of each of a plurality of radiomic features in each of the plurality of pre-diagnostic CT images and in each of the plurality of control CT images; compare the values of the radiomic features in the pre-diagnostic CT images with the values of the radiomic features in the control CT images; based on the comparing, identify a first subset of radiomic features of interest from the plurality of radiomic features, each radiomic feature in the first subset of radiomic features of interest having a change in value from the control CT images to at least a portion of the pre-diagnostic CT images that satisfies a predetermined threshold; and train the machine learning model in conjunction with recursive feature elimination to eliminate at least one radiomic feature from the first subset of radiomic features to form a second subset of radiomic features, the second subset of radiomic features including fewer radiomic features than the first subset of radiomic features, the trained machine learning model outputting a PDAC risk factor based on only the values of the radiomic features in the second subset of radiomic features.

One or more elements or aspects or steps, or any portion(s) thereof, from one or more of any of the claims can be combined with one or more elements or aspects or steps, or any portion(s) thereof, from one or more of any of the other claims or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method for analyzing health of a pancreas of an individual, the method comprising:
receiving a computed tomography (CT) image of a pancreas of the individual;
analyzing the CT image to determine a value of each of one or more radiomic features of the CT image; and
based on the value of each of the one or more radiomic features of the CT image, determining a pancreatic ductal adenocarcinoma (PDAC) risk factor for the pancreas of the individual,
wherein the PDAC risk factor includes an indication of whether a risk of the pancreas of the individual developing a PDAC is low or high, and
wherein the pancreas of the individual includes a plurality of regions, the risk of the pancreas developing a PDAC being low if a risk of each of the plurality of regions developing a PDAC is low, the risk of the pancreas developing a PDAC being high if the risk of at least one of the plurality of regions developing a PDAC is high.

2. The method of claim 1, wherein the PDAC risk factor further includes a probability of the pancreas of the individual developing a PDAC.

3. (canceled)

4. The method of claim 1, wherein the plurality of regions includes a head region, a body region, and a tail region.

5. (canceled)

6. The method of claim 1, wherein the PDAC risk factor further includes (i) an indication of which of the plurality of regions has a highest risk of developing a PDAC, (ii) an indication of which of the plurality of regions will contain a majority of a PDAC if the PDAC develops in the pancreas in the future, or (iii) both (i) and (ii).

7. (canceled)

8. The method of claim 1, wherein the one or more radiomic features of the CT image include (i) a first set of one or more features associated with an intensity of one or more pixels of the CT image, (ii) a second set of one or more features associated with one or more shapes formed by the one or more pixels of the CT image, (iii) a third set of one or more features associated with a variation in intensity of the one or more pixels of the CT image, (iv) a fourth set of one or more features associated with one or more transformations applied to the one or more pixels of the CT image, (v) a fifth set of one or more features associated with one or more filters applied to the one or more pixels of the CT image, (vi) any combination of (i)-(v).

9. The method of claim 1, wherein the one or more radiomic features of the CT image includes a long-run low grey-level emphasis, a short-run low grey-level emphasis, a gaussian left polar, an inverse gaussian left polar, an inverse cluster shade, an inverse cluster prominence, an inverse cluster tendency, or any combination thereof.

10. The method of claim 1, wherein analyzing the CT image and determining the PDAC risk factor includes:
inputting the CT image into a machine learning model; and
receiving the PDAC risk factor as an output of the trained machine learning model,
wherein the machine learning model is trained to analyze the CT image to determine the value of each of the one or more radiomic features of the CT image and determine the PDAC risk factor based on the value of each of the one or more radiomic features of the CT image.

11-13. (canceled)

14. The method of claim 10, wherein the machine learning model is a naive Bayes classifier trained with a training data set that includes a set of feature values obtained from a plurality of training CT images, the set of feature values including values of each of a plurality of features of each of the plurality of training CT images, and wherein the naive Bayes classifier is trained using recursive feature elimination to identify a subset of the plurality of features to be used to determine the PDAC risk factor, the subset of the plurality of features identified during the training forming the one or more radiomic features used to determine the PDAC risk factor for the pancreas of the individual.

15. (canceled)

16. A method of training a machine learning model to analyze pancreas health, the method comprising:
obtaining a plurality of pre-diagnostic computed tomography (CT) images, each of the plurality of pre-diagnostic CT images showing a respective pancreas known to have subsequently developed a pancreatic ductal adenocarcinoma (PDAC);
determining a value of each of a plurality of radiomic features in each of the plurality of pre-diagnostic CT images; and
training the machine learning model, using the value of at least some of the plurality of radiomic features in each of the plurality of pre-diagnostic CT images, to output a PDAC risk factor indicating whether a risk of a PDAC developing in a pancreas of an individual is high or low,
wherein the pancreas of the individual and the respective pancreas of each of the plurality of pre-diagnostic CT images include a plurality of regions, and wherein training the machine learning model includes training the machine learning model to (i) output the PDAC risk factor indicating that the risk of the pancreas of the individual developing a PDAC is high if the risk of at least one of the plurality of regions of the pancreas of the individual developing a PDAC is high, and (ii) output the PDAC risk factor indicating that the risk of the pancreas of the individual developing a PDAC is low if the risk of each of the plurality of regions of the pancreas of the individual developing a PDAC is low.

17-18. (canceled)

19. The method of claim 16, wherein the plurality of radiomic features includes a long-run low grey-level emphasis, a short-run low grey-level emphasis, a gaussian left polar, an inverse gaussian left polar, an inverse cluster shade, an inverse cluster prominence, an inverse cluster tendency, or any combination thereof.

20. The method of claim 16, wherein the plurality of regions includes a head region, a body region, and a tail region.

21. The method of claim 16, wherein determining the value of each of the plurality of radiomic features in each of the plurality of pre-diagnostic CT images includes:
dividing each of the pre-diagnostic CT images into the plurality of regions corresponding to the plurality of regions of the respective pancreas of each of the plurality of pre-diagnostic CT images such that each region of each of the pre-diagnostic CT images corresponds to one of the plurality of regions of the pancreas of the individual; and
determining the value of each of the plurality of radiomic features in each of the plurality of regions of each of the plurality of pre-diagnostic CT images,
wherein training the machine learning model includes training the machine learning model, using the value of at least some of the plurality of radiomic features in each respective region of each of the plurality of pre-diagnostic CT images, to determine the risk of each corresponding region of the pancreas of the individual developing a PDAC.

22-32. (canceled)

33. A method of training a machine learning model to analyze pancreas health, the method comprising:
obtaining a plurality of control computed tomography (CT) images, each of the plurality of control CT images showing a respective pancreas known to have not subsequently developed a PDAC;
obtaining a plurality of pre-diagnostic CT images, each of the plurality of pre-diagnostic CT images showing a respective pancreas known to have subsequently developed a pancreatic ductal adenocarcinoma (PDAC);
determining a value of each of a plurality of radiomic features in each of the plurality of pre-diagnostic CT images and in each of the plurality of control CT images;
comparing the values of the radiomic features in the control CT images with the values of the radiomic features in the pre-diagnostic CT images;
based on the comparing, identifying a first subset of radiomic features of interest from the plurality of radiomic features, each radiomic feature in the subset of radiomic features of interest having a change in value from the control CT images to at least a portion of the pre-diagnostic CT images that satisfies a predetermined threshold; and
training the machine learning model to output a PDAC risk factor using the value of only radiomic features from the first subset of radiomic features in each of the plurality of pre-diagnostic CT images.

34-39. (canceled)

40. The method of claim 33, wherein training the machine learning model includes using recursive feature elimination to eliminate at least one radiomic feature from the subset of radiomic features to form an additional subset of radiomic features, the additional subset of radiomic features including fewer radiomic features than the subset of radiomic features.

41. The method of claim 33, wherein the pancreas and the respective pancreas of each of the plurality of pre-diagnostic CT images and each of the plurality of control CT images include a plurality of regions, and wherein determining the value of each of the plurality of radiomic features in each of the plurality of pre-diagnostic CT images and in each of the plurality of control CT images includes:
dividing each of the pre-diagnostic CT images into the plurality of regions corresponding to the plurality of regions of the respective pancreas of each of the plurality of pre-diagnostic CT images such that each region of each of the pre-diagnostic CT images corresponds to one of the plurality of regions of the pancreas;
dividing each of the control CT images into the plurality of regions corresponding to the plurality of regions of the respective pancreas of each of the plurality of control CT images such that each region of each of the control CT images corresponds to one of the plurality of regions of the pancreas; and
determining the value of each of the plurality of radiomic features in each of the plurality of regions of each of the plurality of pre-diagnostic CT images and in each of the plurality of regions of each of the plurality of control CT images.

42. The method of claim 41, wherein identifying the subset of radiomic features of interest from the plurality of radiomic features includes identifying a subset of radiomic features of interest for each of the plurality of regions, each radiomic feature in the subset of radiomic features of interest for each respective region having a change in value from the respective region of the control CT images to the respective region of the pre-diagnostic CT images that satisfies a predetermined threshold

43. The method of claim 41, further comprising:
obtaining a plurality of diagnostic CT images, each of the plurality of diagnostic CT images corresponding to a respective one of the plurality of pre-diagnostic CT images and showing the respective pancreas after developing the PDAC;
dividing each of the plurality of diagnostic CT images into a plurality of regions corresponding to the plurality of regions of the corresponding one of the plurality of pre-diagnostic CT images such that each region of each of the control CT images corresponds to one of the plurality of regions of the pancreas; and
marking each region of each pre-diagnostic CT image as high-risk or low-risk based on comparing each region of each diagnostic CT image to the corresponding region of the corresponding pre-diagnostic CT image.

44. The method of claim 43, wherein each respective region of each pre-diagnostic CT image is marked as high-risk if (i) at least a portion of the PDAC in the corresponding diagnostic CT image is developed in the respective region or (ii) a majority of the PDAC in the corresponding diagnostic CT image is developed in the respective region.

45. The method of claim 43, wherein each respective region of each pre-diagnostic CT image is marked as low-risk if (i) no portion of the PDAC in the corresponding diagnostic CT image is developed in the respective region or (ii) a minority of the PDAC in the corresponding diagnostic CT image is developed in the respective region.

46. The method of claim 43, further comprising dividing each of the control CT images into a plurality of regions corresponding to the plurality of regions of the respective pancreas of each of the plurality of control CT images such that each region of each of the control CT images corresponds to one of the plurality of regions of the pancreas.

47. The method of claim 46, wherein comparing the values of the radiomic features in the pre-diagnostic CT images to the values of the radiomic features in the control CT images includes, for each respective region of the plurality of regions, comparing (i) the values of the radiomic features in the respective regions of the control CT images and the respective regions marked as low-risk in the pre-diagnostic CT images to (ii) the values of the radiomic features in the respective regions marked as high-risk in the pre-diagnostic CT images.

48. The method of claim 47, wherein each radiomic feature in the subset of radiomic features of interest has a change in value from (i) the respective regions of the control CT images and the respective regions marked as low-risk in the pre-diagnostic CT images to (ii) the respective regions of the pre-diagnostic CT images, where the change in value satisfies a predetermined threshold.

49. The method of claim 46, wherein the plurality of regions includes a head region, a body region, and a tail region, and wherein comparing the values of the radiomic features in the pre-diagnostic CT images to the values of the radiomic features in the control CT images includes:
for the head region, comparing (i) the values of the radiomic features in the head regions of the control CT images and the head regions marked as low-risk in the pre-diagnostic CT images with (ii) the values of the radiomic features in the head regions marked as high-risk in the pre-diagnostic CT images;
for the body region, comparing (i) the values of the radiomic features in the body regions of the control CT images and the body regions marked as low-risk in the pre-diagnostic CT images with (ii) the values of the radiomic features in the body regions marked as high-risk in the pre-diagnostic CT images; and
for the tail region, comparing (i) the values of the radiomic features in the tail regions of the control CT images and the tail regions marked as low-risk in the pre-diagnostic CT images with (ii) the values of the radiomic features in the tail regions marked as high-risk in the pre-diagnostic CT images.

50. The method of claim 49, wherein:
for the head region, each radiomic feature in the first subset of radiomic features of interest has a change in value from (i) the head regions of the control CT images and the head regions marked as low-risk in the pre-diagnostic CT images to (ii) the head regions of the pre-diagnostic CT images, where the change in value satisfies a predetermined threshold;
for the body region, each radiomic feature in the first subset of radiomic features of interest has a change in value from (i) the body regions of the control CT images and the body regions marked as low-risk in the pre-diagnostic CT images to (ii) the body regions of the pre-diagnostic CT images, where the change in value satisfies the predetermined threshold; and
for the tail region, each radiomic feature in the first subset of radiomic features of interest has a change in value from (i) the tail regions of the control CT images and the tail regions marked as low-risk in the pre-diagnostic CT images to (ii) the tail regions of the pre-diagnostic CT images, where the change in value satisfies the predetermined threshold.
